(19) United States
(12) Patent Application Publication
(10) Pub. No.: US 2015/0010945 A1
(43) Pub. Date: Jan. 8, 2015

(54) METHODS AND SYSTEMS FOR TRACKING BIOREMEDIATION PROCESSES

(71) Applicants: Rosa Krajmalnik-Brown, Chandler, AZ (US); Rolf U. Halden, Phoenix, AZ (US); James W. Wilson, Drexel Hill, PA (US)

(72) Inventors: Rosa Krajmalnik-Brown, Chandler, AZ (US); Rolf U. Halden, Phoenix, AZ (US); James W. Wilson, Drexel Hill, PA (US)

(73) Assignee: Arizona State Board of Regents For and on Behalf of Arizona State University, Scottsdale, AZ (US)

(21) Appl. No.: 14/127,298

(22) PCT Filed: Jun. 19, 2012
(86) PCT No.: PCT/US12/43174

§ 371 Date: Mar. 20, 2014

Related U.S. Application Data
(60) Provisional application No. 61/501,020, filed on Jun. 24, 2011.

(51) Int. Cl. C12N 15/70; H01J 49/00; H01J 49/04; C07K 14/195

(52) U.S. Cl. CPC C12N 15/70; C07K 14/195; H01J 49/0031; H01J 49/04
USPC 435/69.1; 435/471; 435/320.1; 250/282

(57) ABSTRACT

A method for expression of engineered constructs containing sequences coding for functional genes of interest in E. coli strains. Here tceA and tceB are fused to an inducible, active promoter to optimize transcription of the open reading frames, and an introduced consensus Shine-Dalgarno sequence allowing for optimal ribosome binding and translation of the open reading frames, and codons optimized for expression in E. coil. This over-expressed protein is then used to design appropriate mass spectro metric methods for environmental detection.

### CROSS-REFERENCE TO RELATED APPLICATIONS

Related technology is disclosed in pending PCT patent application publication WO2011/011683, published on Jan. 27, 2011, and entitled “MICROBIAL CULTURES AND METHODS FOR ANAEROBIC BIOREMEDIATION,” to co-inventors of the present application, of which the entire content is incorporated herein by reference.

### TECHNICAL FIELD

The present invention relates to methods and systems for tracking bioremediation processes. More particularly, the invention relates to methods for the convenient expression of genes of Dehalococcoides and other relevant microorganisms for remediation and bioenergy and the mass spectrometric detection of gene expression products for tracking the progress of bioremediation and bioenergy related efforts during cleanup of chloroethene-contaminanted environments.

The present invention shows an example of how this invention is applied to detect and track a reductive dehalogenase enzyme in Dehalococcoides i.e, TceA, however this over expression and detection system can be applied to track many other enzymes of interest.

### BACKGROUND

Microorganisms of the family Dehalococcoides are known to perform the beneficial biotransformation of toxic chlorinated ethenes to non-toxic ethene at hazardous waste sites. At the community level, Dehalococcoides performance can be improved by the presence of homoacetogens, where the presence of methanogens diverts electrons and slows down dechlorination. Quantification of Dehalococcoides sp. using isolated DNA and quantitative PCR [1-2] has become one of multiple lines of evidence for remediation decision making; however, the information obtained based on these DNA-based assays is limited. The current assays target 3 identified RDases tceA [3], bvcA [4], and vcrA [5]. Two key limitations are: 1. The primers can be too specific and therefore may not fully capture homolog proteins, which serve the same function but are slightly different at the gene level, and 2. Quantifying Dehalococcoides genes provides no information on microbial interactions, and how these interactions affect dechlorination rates.

The present invention overcomes these two limitations. The disclosed methods and systems have the potential to enhance or even replace existing procedures and materials for the determination of bioremediation progress. The technology disclosed herein overcomes existing limitations in the availability and production of proteins for use as bioremediation agents and as diagnostic materials. The instant over-expression method will allow development of more specific and sensitive protein detection methods which are helpful to detect Dehalococcoides enzymes but could also be applied to many other systems.

### BRIEF SUMMARY OF THE DISCLOSURE

This summary is provided to introduce, in a simplified form, a selection of concepts that are further described below in the Detailed Description. This summary is not intended to identify key features of the claimed subject matter, nor is it intended to be used as an aid in determining the scope of the claimed subject matter.

A method of over-expressing proteins originating in Dehalococcoides where gene expression products are separated (e.g., on an SDS gel), and where the gene expression products include proteins encoded by a pBAD18 +a gene of interest, such as, for example, a tceAB construct. The protein gels are refrigerated at 4° C. and later used for de novo sequencing of peptides using proteomic mass spectrometry.

In one aspect, the gel comprises a 5-20% SDS-polyacrylamide gel.

In another aspect a mass spectrometric method for tracking the expression of genes of Dehalococcoides includes the steps of acquiring an environmental sample, extracting proteins and peptides contained therein, introducing proteins and peptides originating from said sample into a mass spectrometer, and analyzing the resultant mass spectra by utilizing reference spectra from overexpressed proteins and peptides from Dehalococcoides.

In another aspect the method is modified by the use of labeled proteins and peptides for target quantification.

In another aspect the method is modified by using isotope labeled peptides for (absolute) quantitation of target proteins and peptides.

In another aspect, a mass spectrometric method for tracking the expression of genes of Dehalococcoides includes acquiring an environmental sample, extracting proteins and peptides contained therein, introducing proteins and peptides originating from said sample into a mass spectrometer, and monitoring ions featuring a mass-to-charge ratio (m/z) specific to peptides resulting from protein digestion with a suitable enzyme (e.g., trypsin) of expression products of the target genes of interest (e.g., tceA [3], bvcA [4], and vcrA).

In another aspect a method for expression of engineered constructs containing sequences coding for functional genes of interest in E. coli strains includes fusing tceA and tceB to an inducible, active promoter to optimize transcription of the open reading frames, and an introduced consensus Shine-Dalgarno sequence allowing for optimal ribosome binding and translation of the open reading frames, and codons optimized for expression in E. coil.

In another aspect, a method of utilizing the metabolic capability of a living organism, with the method includes (a) performing codon optimization of the gene sequence to be expressed for the specific expressing host; (b) using a transforming vector; (c) adding a strong promoter and ribosomal binding sequences for increased expression of the protein of interest; and (d) detecting the over expressed protein using mass spectrometric methods.

### BRIEF DESCRIPTION OF THE DRAWINGS

While the novel features of the invention are set forth with particularity in the appended claims, the invention, both as to organization and content, will be better understood and appreciated, along with other objects and features thereof, from the following detailed description taken in conjunction with the drawings, in which:

FIG. 1 schematically illustrates a prior art plasmid construct map of the gene TceAB (50997-3) cloned in pBAD18 by EcoRI and KpnI.

FIG. 2 schematically illustrates a pBAD18 cloning map.

FIG. 3 and FIG. 3A graphically illustrate the mass spectrometric analysis of an over-expressed gene product, i.e., an RDase (specifically TceA here), using Matrix Assisted Laser Desorption Ionization/Tandem Time-Of-Flight mass spectrometry (MALDI/TOF/TOF). The spectrum reveals specific peptides suitable for mass spectrometric tracking by their characteristic mass-to-charge ratios. To obtain the spectrum, the gene expression product was digested with trypsin and analyzed by MALDI-TOF/TOF. The resultant spectrum was analyzed with standard proteomics software to reveal the identity and structure of peptides corresponding to the mass-to-charge ratios observed.

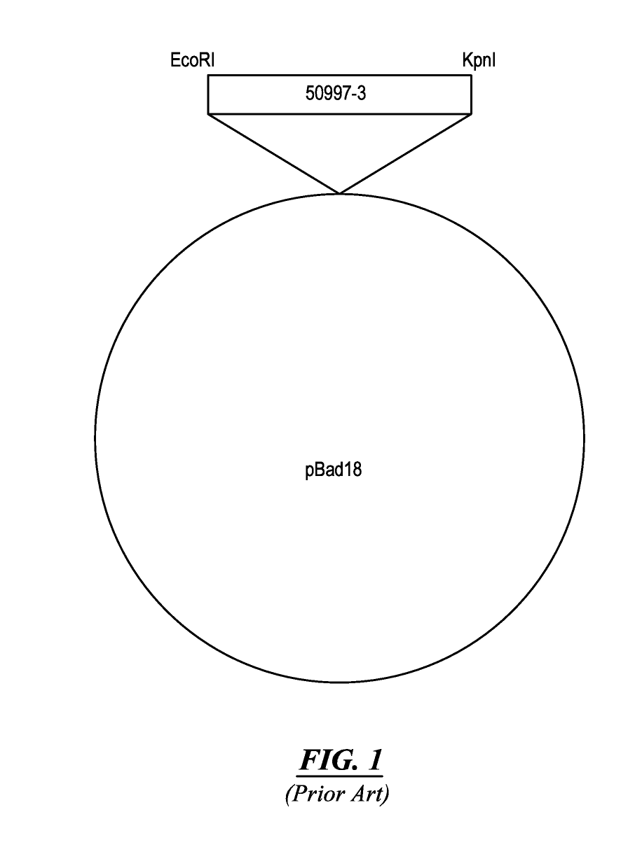

In the drawings, identical reference numbers identify similar elements or components. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

### DETAILED DESCRIPTION OF THE PREFERRED EMBODIMENTS

The following disclosure describes tracking bioremediation processes. Several features of methods and systems in accordance with example embodiments are set forth and described in the FIG.s. It will be appreciated that methods and systems in accordance with other example embodiments can include additional procedures or features different than those shown in the FIG.s. Example embodiments are described herein with respect to analysis of environmental conditions. However, it will be understood that these examples are for the purpose of illustrating the principles, and that the invention is not so limited. Additionally, methods and systems in accordance with several example embodiments may not include all of the features shown in the FIG.s.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one example” or “an example embodiment,” “one embodiment,” “an embodiment” or combinations and/or variations of these terms means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### DEFINITIONS

Generally, as used herein, the following terms have the following meanings when used within the context of contaminant sample collection in aquatic or saturated sedimentary environments:

A “sample” as used herein refers to material, such as environmental material obtained from a remediation site that is suspected of containing, or known to contain, an analytes, as for example, proteins and peptides.

“EcoRI” is a well known endonuclease enzyme isolated from strains of E. coli, and is part of the restriction modification system used in cloning.

“KpnI” is a well known restriction enzyme.

The term “TOP10 strain” as used herein refers to E. coli competent TOP10 strains as supplied for example, by Invitrogen Corporation, now part of Life Technologies of California, US.

The term “TCE” as used herein refers to trichloroethene.

tceA is the gene that encodes for the enzyme TceA. TceA performs the dechlorination (conversion to less chlorinated compounds) of TCE.

RDase is a/any reductive dehalogenase, these are enzymes that convert chlorinated compounds to less chlorinated products.

### EXAMPLE EMBODIMENTS

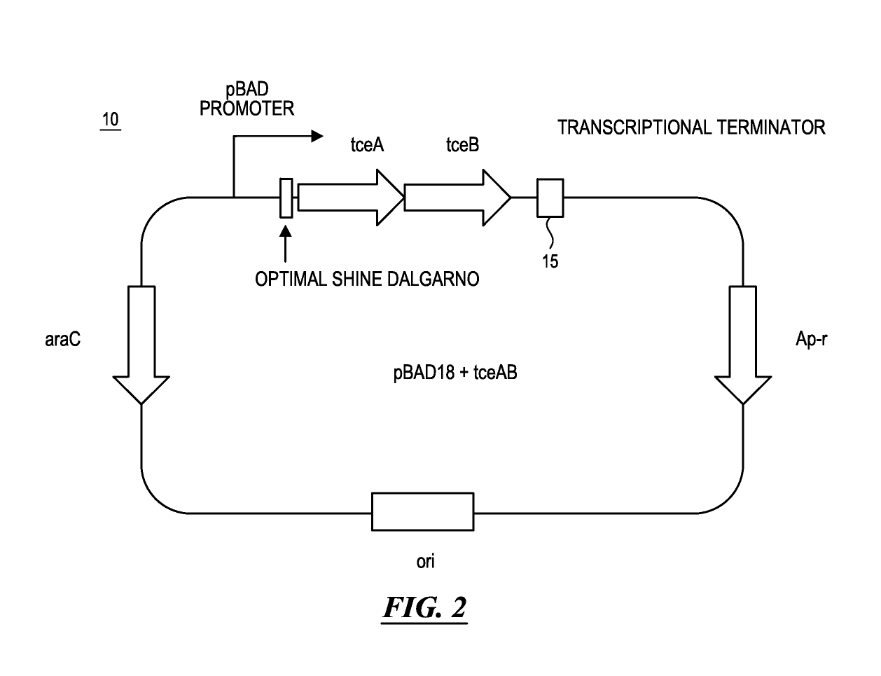

Referring now to FIG. 2, a pBAD18 cloning map is schematically illustrated for designing, cloning and expression of engineered constructs containing sequences coding for functional genes of interest (in this case tceA and tceB) in E. coli strains. A gene construct 10 contains optimal expression signals for a functional tceA tceB cassette. The construct 10 comprises the products of fusion of the above genes to an inducible, highly active promoter to optimize transcription of the open reading frames, and an introduced consensus Shine-Dalgarno sequence allowing for optimal ribosome binding and translation of the open reading frames, and codons optimized for expression in E. coil. These features greatly enhance the ability to obtain enhanced expression of the enzyme of interest, in this case the TceA and TceB enzymes.

In one example, the 1792-bp section containing the two genes of interest tceA and tceB was cloned downstream of the arabinose-inducible promoter in plasmid pBAD18. The pBAD18 cloning map includes an optimal Shine Dalgarno sequence followed by genes tceA and tceB and a transcriptional terminator (15 in FIG. 2). The codon optimization for TceAB over expression in E. coli is listed as SEQ. NO. 1 in the appended sequence listing. The remaining DNA sequences in the expression vector comprise the E. coli DNA for the pBAD18 cloning vector sequence as detailed below and in the appended sequence listing:

E. coli DNA for pBAD18 Cloning Vector (SEQ. NO. 2):
GenBank: X81838.1
LOCUS X81 838 461 3 by DNA circular SYN 25 Oct. 1995
DEFINITION E. coli DNA for pBAD18 cloning vector.
ACCESSION X81838
VERSION X81838.1 GI:551423
KEYWORDS arac gene; bla gene; expression vector.
SOURCE synthetic construct
   ORGANISM synthetic construct
      other sequences; artificial sequences.
REFERENCE 1 (bases 1 to 4613)
   AUTHORS Guzman, L. M., Belin, D., Carson, M. J. and Beckwith, J.
   TITLE Tight regulation, modulation, and high-level expression by Vectors containing the arabinose PBAD promoter
   JOURNAL J. Bacteriol. 177 (14), 4121-4130 (1995)
   PUBMED 7608087
REFERENCE 2 (bases 1 to 4613)
   AUTHORS Guzman, L. M.
   TITLE Direct Submission
   JOURNAL Submitted (22-SEP-1994) L. M. Guzman, Harvard Medical School, Dept of M Ave., Boston Mass. 02115, USA
COMMENT Related sequences: J01641 and M10196 (bases 1-1300); M77749 (bases 1357-2296; 4537-4613); SYNPBR322 (bases 2297-2780); Gene 27, 183, 1984 (bases 2780-3238).

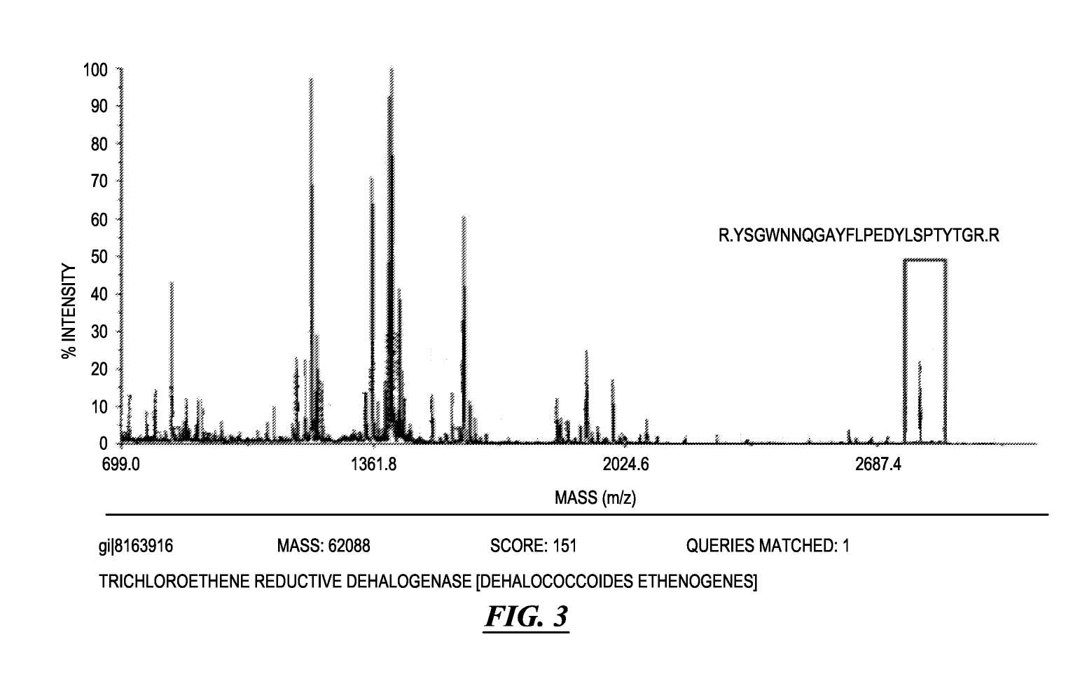

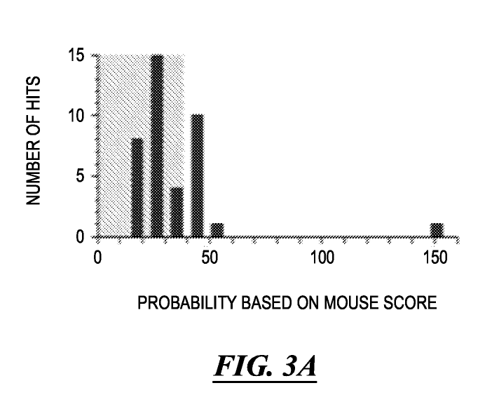

Referring now to FIG. 3 and FIG. 3A, a confirmatory analysis of successful TceA expression is shown here using Matrix Assisted Laser Desorption Ionization/Tandem Time-Of-Flight (MALDI/TOF/TOF) mass spectrometry. Gene expression products were separated on an SDS gel. In one example, expression of the protein encoded on the BAD18+tceAB construct was verified using a 12% SDS-polyacrylamide gel. The protein gel was refrigerated at 4° C. and later used for de novo sequencing of peptides using proteomic mass spectrometry. The corresponding band was extracted and analyzed by peptide mass fingerprinting and peptide mass sequencing using MALDI-TOF/TOF. This represents the first successful over-expression of this TCE reductive dehalogenase (TCE-RDase). The disclosed process demonstrates an ability to generate large quantities of key enzymes of the slow-growing Dehalococcoides, a feat previously unattained when using current state-of-the art methods.

The above over-expressed proteins were used to develop a sensitive proteomic detection approach. Proteomic assays will make use of isotope-labeled peptides to be custom-synthesized based on the peptide mass fingerprints and ionization properties of digested target RDases. These novel proteomic assays will employ the isotope dilution technique for absolute quantitation of proteins in environmental samples (AQUA). Using the capsid protein of the Norovirus (“cruise ship virus”) as a target, we have demonstrated that the use of electrospray and MALDI tandem mass spectrometry allows for the detection of attomole levels of target protein (1 atto mole=1×10⁻¹⁸ moles) [6].

The invention has been described herein in considerable detail in order to comply with the Patent Statutes and to provide those skilled in the art with the information needed to apply the novel principles of the present invention, and to construct and use such exemplary and specialized components as are required. However, it is to be understood that the invention may be carried out by different equipment, and devices, and that various modifications, both as to the equipment details and operating procedures, may be accomplished without departing from the true spirit and scope of the present invention.

### REFERENCES

The teachings of the following publications are incorporated herein in their entirety by this reference.
[1] Werner, J. J., A. C. Ptak, B. G. Rahm, S. Zhang, and R. E. Richardson, Absolute quantification of Dehalococcoides proteins: enzyme bioindicators of chlorinated ethene dehalorespiration. Environmental Microbiology, 2009. 11(10): p. 2687-2697.
[2] Ritalahti, K. M., B. K. Amos, Y. Sung, Q. Z. Wu, S. S. Koenigsberg and F. E. Loffler (2006). “Quantitative PCR targeting 16S rRNA and reductive dehalogenase genes simultaneously monitors multiple Dehalococcoides strains.” Applied and Environmental Microbiology 72(4): 2765-2774.

[3] Magnuson, J. K., M. F. Romine, D. R. Burris, and M. T. Kingsley. 2000. Trichloroethene reductive dehalogenase from Dehalococcoides ethenogenes: sequence of tceA and substrate range characterization. Appl. Environ. Microbiol. 66:5141-5147.

[4] Krajmalnik-Brown R., T. Hölscher, I. N. Thomson, F. M. Saunders, K. M. Ritalahti, and F. E. Löffler. 2004 “Genetic Identification of a Putative Vinyl Chloride Reductase in Dehalococcoides sp. Strain BAV1”. Applied and Environmental Microbiology. 70(10): 6347-6351.

[5] Müller, J. A., B. M. Rosner, G. von Abendroth, G. Meshulam-Simon, P. L. McCarty, and A. M. Spormann. 2004. Molecular identification of the catabolic vinyl chloride reductase from Dehalococcoides sp. strain VS and its environmental distribution. Appl. Environ. Microbiol. 70:4880-4888.

[6] Colquhoun, D. R., Schwab, K. J., Cole, R. N., and R. U. Halden. 2006. Detection of Norovirus Capsid Protein in Authentic Standards and in Stool Extract by Matrix-Assisted Laser Desorption Ionization and Nanospray Mass Spectrometry. Appl. Environ. Microbiol. 72(4):2749-2755.

What is claimed is:

1. A method for expression of engineered constructs containing sequences coding for functional genes of interest in E. coli strains comprising:
fusing genes of interest to an inducible, active promoter to optimize transcription of the open reading frames, and an introduced consensus Shine-Dalgarno sequence allowing for optimal ribosome binding and translation of the open reading frames, and codons optimized for expression in E. coil.

2. The method of claim 1 wherein the step of fusing includes cloning tceA and tceB downstream of the arabinose-inducible promoter in plasmid pBAD18.

3. A method for expression of engineered constructs containing sequences coding for Reductive Dehalogenase enzymes (RDase) in E. coli strains comprising:
fusing genes of interest to an inducible, active promoter to optimize transcription of the open reading frames, and an introduced consensus Shine-Dalgarno sequence allowing for optimal ribosome binding and translation of the open reading frames, and codons optimized for expression in E. coil.

4. The method of claim 3 wherein the step of fusing includes cloning tceA and tceB downstream of the arabinose-inducible promoter in plasmid pBAD18.

5. A method for expression of engineered constructs containing sequences coding for functional genes of interest in E. coli strains comprising:
fusing the genes of interest (i.e., tceA and tceB) to an inducible, active promoter to optimize transcription of the open reading frames, and an introduced consensus Shine-Dalgarno sequence allowing for optimal ribosome binding and translation of the open reading frames, and codons optimized for expression in E. coil.

6. The method of claim 5 wherein the step of fusing includes cloning tceA and tceB downstream of the arabinose-inducible promoter in plasmid pBAD18.

7. An engineered construct containing sequences coding for functional genes of interest in E. coli strains comprising:
genes of interest (i.e., tceA and tceB) fused to an inducible, active promoter to optimize transcription of the open reading frames, and an introduced consensus Shine-Dalgarno sequence allowing for optimal ribosome binding and translation of the open reading frames, and codons optimized for expression in E. coil.

8. The construct of claim 7 wherein tceA and tceB are cloned downstream of the arabinose-inducible promoter in plasmid pBAD18.

9. A method of overexpressing proteins originating in Dehalococcoides or other difficult to grow anaerobic microorganisms, with the method comprising:
separating products of gene expression, where the gene expression products include proteins encoded on a pBAD18+gene of interest construct; and
using the separated proteins for de novo sequencing of peptides by employing proteomic mass spectrometry.

10. The method of claim 9 wherein separating products of gene expression comprises using SDS gel.

11. The method of claim 10 wherein the SDS gel comprises a 5-20% SDS-polyacrylamide gel.

12. A mass spectrometric method for tracking the expression of genes of Dehalococcoides or other difficult to grow anaerobic microorganisms, comprising the steps of:
acquiring an environmental sample;
extracting proteins and peptides contained therein;
introducing proteins and peptides originating from said sample into a mass spectrometer; and
analyzing the resultant mass spectra by utilizing reference spectra from overexpressed proteins and peptides from Dehalococcoides.

13. The method of claim 12 wherein the method further comprises using labeled proteins and peptides for target quantification.

14. The method of claim 12 wherein the method further comprises using isotope labeled peptides for protein and peptide quantification.

15. A mass spectrometric method for tracking the expression of genes of Dehalococcoides, comprising the steps of:
acquiring an environmental sample and extracting proteins and peptides contained therein;
introducing proteins and peptides originating from said sample into a mass spectrometer; and
monitoring ions of digestion products by utilizing their characteristic mass-to-charge ratios (m/z).

16. The method of claim 15 wherein the digestion products comprise peptides.

17. A method of utilizing the metabolic capability of a living organism, with the method comprising:
(a) performing codon optimization of the gene sequence to be expressed for the specific expressing host;
(b) using a transforming vector;
(c) adding a strong promoter and ribosomal binding sequences for increased expression of the protein of interest; and
(d) detecting the over expressed protein using mass spectrometric methods.


SEQUENCE LISTING (2 sequences)

SEQ ID NO: 1 - DNA, 2018 bp, Artificial Sequence
  Related sequences J01641 and M10196 (bases 1-1300); M77749 (bases 1357-2296; 4537-4613); SYNPBR322 (bases 2297-2780); Gene 27, 183, 1984 (bases 2780-3238).
tgggctagcgaattcaggaggacagctatgagtgaaaaatatcattcaactgttacccggcgtgattttatgaaacgtttagggctggccggggccggcgctggcgcactgggcgcggcagtgcttgcggaaaacaacttaccgcatgaatttaaggatgtcgatgatctgctgtcagccggtaaagccttggagggtgatcacgctaataaagtgaacaaccatccgtggtgggtgaccacccgtgatcatgaagatccgacgtgcaacatcgattggtcgctgattaaacgttactcgggttggaacaaccaaggtgcctatttcctgccggaagattacttgagtccaacttataccggcagacgtcatacgattgtggatagcaaattagaaattgaattacagggtaagaagtaccgtgattccgcgtttattaaaagtggtatcgactggatgaaagagaacattgacccggattatgatccaggggaactgggctacggcgatcgccgtgaagatgctcttatctacgccgctactaacggttcccacaattgttgggaaaaccctctttatggccgctatgaaggtagccgtccgtacctgagtatgcgtaccatgaacggcattaatggcctccatgaatttggtcatgcagacatcaagacgactaactacccgaaatgggagggtacgccggaagaaaaccttctgataatgcggaccgcggcccgctattttggcgcctcatccgtcggtgctatcaagataaccgataacgtgaaaaagattttctatgccaaagtgcaaccgttttgtctgggcccgtggtacaccatcaccaacatggccgaatatattgaatatccggtgccggttgacaattacgccattccgattgtgtttgaagacattccggccgatcaaggtcattattcgtataagcgcttcggtggtgatgataaaattgctgtgccgaacgcccttgataacatattcacgtacacgatcatgctgccagaaaaacgttttaaatacgcgcattcgatcccgatggatccgtgttcttgcattgcttacccgctgttcacagaagttgaagctaggattcagcaatttattgcgggcctgggttataattccatgggtggcggggttgaagcgtgggggccgggcagcgcttttggaaacctatcggggctgggggaacagtcgcgtgtcagcagcatcatcgagccgcgttatggatcaaataccaaaggttcactgcggatgctgacagacctgccgctcgcaccgactaagccaatcgacgcgggcatccgcgagttctgtaaaacttgcggcatctgcgcggaacactgcccgactcaagcaatctctcacgaaggcccgcggtatgactctccccattgggattgtgttagcggttacgaaggttggcacttagattatcataaatgtattaactgcaccatctgtgaagctgtttgcccattctttaccatgagtaataatagctgggttcataatctggtgaaaagcaccgtcgccactacgccggtttttaatggctttttcaaaaatatggaaggcgcgtttggctatggtccccgatatagtccgagccgtgatgagtggtgggctagtgaaaatccaattcggggcgcgagcgttgatatcttttaagagaaaggatggaatagattattatgggcggggctctgtattatttcctggttggaatgctgattggtggtgccgccatttggtttattacctatacccagtttaaaaatattagctttaagtggtgggaatggtctctgatggcactgtcactgctgctggtcagtagcatctttcaacacatgtattcttcaatgagcgtggaaatggaatatcagagcgcatttatgtatctgggcgtgtttggtactctggccgtgatcctaaacctgatcgtgtggcgtacgtatagcggcagaaaagaataaaatggtaccgcatgcaac

SEQ ID NO: 2 - DNA, 4613 bp, Artificial sequence
  Related sequences J01641 and M10196 (bases 1-1300); M77749 (bases 1357-2296; 4537-4613); SYNPBR322 (bases 2297-2780); Gene 27, 183, 1984 (bases 2780-3238).
atcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattcgagctcggtacccggggatcctctagagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatc